(19) United States
(12) Patent Application Publication
(10) Pub. No.: US 2007/0299282 A1
(43) Pub. Date: Dec. 27, 2007

(54) Method for the Production of Phthalic Dichloride

(75) Inventors: Thorsten Rohde, Mannheim (DE); Jochem Henkelmann, Mannheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/719,983

(22) PCT Filed: Nov. 24, 2005
(86) PCT No.: PCT/EP05/12564

§ 371 Date: May 23, 2007

(30) Foreign Application Priority Data
Nov. 26, 2004 (DE) 10 2004 057 146.5

(51) Int. Cl. C07C 51/60

(52) U.S. Cl. 562/855

(57) ABSTRACT

A process for preparing phthaloyl chloride by reacting phthalic anhydride with a chlorinating agent (I) selected from the group of thionyl chloride and phosgene in the presence of a catalyst at a temperature of from 80 to 200° C. and a pressure of from 0.01 to 10 MPa abs, in which the catalyst (II) used is triphenylphosphine, triphenylphosphine oxide or a mixture thereof.

The present invention relates to a process for preparing phthaloyl chloride by reacting phthalic anhydride with a chlorinating agent (I) selected from the group of thionyl chloride and phosgene in the presence of a catalyst at a temperature of from 80 to 200° C. and a pressure of from 0.01 to 10 MPa abs.

Phthaloyl chloride is an important starting material in the preparation of plasticizers and synthetic resins. In addition, phthaloyl chloride is also used as a synthesis building block in the preparation of active ingredients, for example of pharmaceuticals and crop protection agents.

Phthaloyl chloride is prepared generally by reacting phthalic anhydride with suitable chlorinating agents.

O. Gräbe in Liebigs Ann., 1887, page 318 to 337 (footnote page 329) and S. Wolfe et al. in Canadian Journal of Chemistry 48, 1970, page 3566 to 3571 disclose the preparation of phthaloyl chloride by reacting phthalic anhydride with phosphorus(V) chloride. A disadvantage of this process is the only low yield of 54% according to S. Wolfe et al., Canadian Journal of Chemistry 48, 1970, page 3570, and the formation of stoichiometric amounts of phosphorus oxide trichloride as a coproduct, which has to be removed from the phthaloyl chloride product of value and is costly and inconvenient to dispose of. In addition, phosphorus(V) chloride is a solid under standard conditions and is therefore industrially more difficult to handle than, for example, a liquid or a gas.

U.S. Pat. No. 2,051,096 describes the preparation of phthaloyl chloride by reacting phthalic anhydride with trichloromethane or tetrachloromethane in the presence of zinc chloride. This process has a series of disadvantages. For instance, the use of chlorinated hydrocarbons is quite problematic from the current environmental policy point of view. In addition, the process specified requires a high reaction temperature of from about 220 to 300° C.

In addition, owing to the use of a Lewis acid containing a metal cation (zinc chloride), the subsequent workup of the resulting reaction mixture is costly and inconvenient, since, after the phthaloyl chloride product of value has been distilled off, a mixture remains in the bottom which comprises the Lewis acid used and the organic by-products formed. This cannot simply be disposed of, but rather has to be separated in a further costly and inconvenient step into the Lewis acid containing a metal cation and the organic by-products. The Lewis acid containing a metal cation and the organic by-products then have to be disposed of separately.

DE-A 20 36 171 teaches the preparation of phthaloyl chloride by reacting phthalic anhydride with trichloromethyl isocyanate dichloride in the presence of a Lewis acid, in particular zinc chloride, iron(III) chloride or aluminum chloride. A disadvantage of this process is the formation of stoichiometric amounts of chlorocarbonyl isocyanide dichloride and fumaryl chloride as coproducts which have to be removed from the phthaloyl chloride product of value and have to be disposed of in a costly and inconvenient manner. In addition, the workup of the resulting reaction mixture is costly and inconvenient owing to the use of the Lewis acids containing metal cations (see above).

L. P. Kyrides, J. Am. Chem. Soc. 59, 1937, page 206 to 208 describes the preparation of phthaloyl chloride by reacting phthalic anhydride with benzotrichloride in the presence of zinc chloride. A disadvantage of this process is the formation of stoichiometric amounts of benzoyl chloride as a coproduct which has to be removed from the phthaloyl chloride product of value and is costly and inconvenient to dispose of. In addition, the workup of the resulting reaction mixture is costly and inconvenient owing to the use of zinc chloride (see above).

A common disadvantage of the abovementioned processes is the mentioned formation of stoichiometric amounts of coproduct which remains in the reaction mixture and has to be removed from the phthaloyl chloride product of value and is costly and inconvenient to dispose of. When thionyl chloride or phosgene is used as the chlorinating agent, this disadvantage does not occur, since only gaseous by-products are formed from the chlorinating agent in this case, specifically hydrogen chloride gas, sulfur dioxide or carbon dioxide, which are easy to remove.

Thus, L. P. Kyrides, J. Am. Chem. Soc. 59, 1937, page 206 to 208 also teaches the preparation of phthaloyl chloride by reacting phthalic anhydride with thionyl chloride in the presence of zinc chloride. Compared to the abovementioned processes, the process mentioned at least has the advantage that the thionyl chloride chlorinating agent to be used only forms gaseous by-products which are easy to remove. Nevertheless, this process has crucial disadvantages. For instance, the required reaction temperature of 220° C. is very high and the achievable yield of 86% only moderate (see experimental section in L. P. Kyrides), even if an estimation of the space-time yield achieved (see definitions section) gives a respectable value of about 73 g/l·h. In addition, the workup of the resulting reaction mixture is costly and inconvenient owing to the use of zinc chloride (see above).

U.S. Pat. No. 3,810,940 and DE-A 102 37 579 teach the preparation of phthaloyl chloride by reacting phthalic anhydride with phosgene in the presence of an N,N-dialkylformamide and in the presence of an inert solvent. U.S. Pat. No. 3,810,940 teaches specifically the use of N,N-dimethylformamide as a catalyst and of chlorobenzene as a solvent. These processes too have the advantage that the phosgene chlorinating agent to be used forms only gaseous by-products which are easy to remove. Nevertheless, these processes also show crucial disadvantages. For instance, the estimated space-time yields achievable with a range of from 31 to 52 g/l·h (DE-A 102 37 579 Example 1: about 42 g/l·h, Example 2: about 31 g/l·h, Example 3: about 32 g/l·h; U.S. Pat. No. 3,810,940 Example 4: about 52 g/l·h) are very low and thus absolutely unsatisfactory. In addition, the use of N,N-dialkylformamides as a catalyst, as a result of reaction with phosgene, forms amide-hydrochloride adducts, known as Vilsmeier adducts, which are of ionic nature and can therefore lead to problems owing to solid precipitation in the subsequent distillation. In addition, the amide-hydrochloride adducts formed can thermally decompose in the subsequent distillation and lead to contamination of the phthaloyl chloride product of value.

DE-A 40 06 370 discloses the general teaching for the synthesis of aliphatic, aromatic or araliphatic carbonyl chlorides from phosgene and the corresponding carboxylic acids or carboxylic anhydrides in the presence of dialkylated carboxamides and/or trisubstituted phosphine oxides and/or sulfides as phosgenation catalysts and in particular of dimethylformamide, dimethylacetamide and trioctylphosphine oxide.

It is an object of the present invention to find a process for preparing phthaloyl chloride of high purity, which does not have the abovementioned disadvantages, requires only readily obtainable and industrially widely available reactants and, if appropriate, only readily obtainable and industrially widely available assistants/catalysts, and the reactants to increase the space-time yield and to enable a technically less costly and inconvenient isolation of the phthaloyl chloride product of value do not form any coproducts which remain predominantly in the reaction mixture. In addition, the phthaloyl chloride product of value should be readily removable in the process to be found from any assistants/catalysts required and from any intermediates and by-products obtained, any assistants/catalysts required and their possible reaction products should not tend to solid precipitation under the present conditions, and it should be possible to dispose of any assistants/catalysts required without great cost and inconvenience. In addition, even under mild temperatures and pressures, the process to be found should also lead to a high conversion of phthalic anhydride, a high selectivity for and a high yield of phthaloyl chloride, and in particular a high space-time yield.

Accordingly, a process has been found for preparing phthaloyl chloride by reacting phthalic anhydride with a chlorinating agent (I) selected from the group of thionyl chloride and phosgene in the presence of a catalyst at a temperature of from 80 to 200° C. and a pressure of from 0.01 to 10 MPa abs, which comprises using as the catalyst (II) triphenylphosphine, triphenylphosphine oxide or a mixture thereof.

The triphenylphosphine, triphenylphosphine oxide or mixture thereof to be used as the catalyst (II) in the process according to the invention is characterized by the common structural formula (IIa) 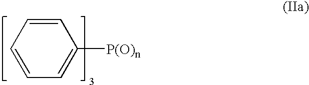
where n is 0 or 1.

It has been recognized in the context of the invention that other phosphines, for example trialkylphosphines and trialkylphosphine oxides, lead to dramatically lower conversions, yields and space-time yields (on this subject, see comparative example 10).

The amount of catalyst (II) to be used in the process according to the invention is generally from 0.05 to 25 mol %, preferably from 0.1 to 20 mol %, more preferably from 0.2 to 10 mol % and most preferably from 0.5 to 5 mol %, based on the phthalic anhydride used.

The chlorinating agent (I) used in the process according to the invention is thionyl chloride or phosgene. The molar ratio of chlorinating agent (I) used is generally from 0.95 to 10, preferably from 0.95 to 5, more preferably from 0.98 to 3 and most preferably from 1 to 2, based on the molar amount of phthalic anhydride used.

In the process according to the invention, preference is given to using thionyl chloride as the chlorinating agent (I).

The phthalic anhydride, the chlorinating agent (I), the catalyst (II) and any solvent to be used may be added in various ways and in various sequences.

The phthalic anhydride may be used, for example, in molten form or dissolved in an inert solvent. In addition, it is possible to dissolve a portion or the entire amount of catalyst (II) in the phthalic anhydride, if appropriate in the presence of an additional inert solvent, and to add it in this way.

When thionyl chloride is used, the chlorinating agent (I) is added generally in liquid form and, when phosgene is used, generally in gaseous form. Depending on the case, it is, however, also possible to add phosgene in liquid form. In addition, it is also possible in principle to dissolve a portion or the entire amount of catalyst (II) in the chlorinating agent, if appropriate in the presence of an additional inert solvent, and to add it in this way. When thionyl chloride is used, preference is given to dissolving a portion of the catalyst (II) therein and to feeding it in this form to the phthalic anhydride.

Alternatively to the abovementioned addition methods for the catalyst (II), it may also be added separately, dissolved in the required amount of inert solvent.

The process according to the invention is generally operated semicontinuously or continuously. In semicontinuous mode, phthalic anhydride is typically initially charged in a suitable reaction apparatus in the molten state or dissolved in an inert solvent, and the entire amount of catalyst (II) or at least a portion thereof is dissolved therein. Subsequently, the chlorinating agent (I) which if appropriate comprises the remaining amount of the catalyst (II) and if appropriate an inert solvent is added continuously according to the progress of the reaction.

In the continuous mode, the reactants and the catalyst (II) which are if appropriate dissolved in an inert solvent are typically fed simultaneously to a suitable reaction apparatus, in the course of which an amount corresponding to the amount fed is removed simultaneously from the reaction apparatus.

Suitable reaction apparatus is in principle all reaction apparatus which is suitable for gas/liquid or liquid/liquid reactions, for example stirred tanks.

Inert solvents refer to solvents which do not react chemically with the substances mentioned under the selected conditions. Preference is given to using aromatic or aliphatic hydrocarbons and phthaloyl chloride (m.p. 12° C.). The latter has the advantage that no further extraneous substances are introduced into the process as a result. When inert solvents are used, preference is given to selecting those which, to avoid evaporative cooling, have a higher boiling point than the chlorinating agent (I) used and additionally, for better removability of the phthaloyl chloride in the distillative workup, have a boiling point lower by preferably at least 10° C., measured at standard pressure, than phthalic anhydride. However, it is alternatively also possible to use inert solvents which, for sufficient removability of the phthaloyl chloride, have a boiling point higher by preferably at least 10° C., measured at standard pressure, than phthaloyl chloride. Preferred hydrocarbons are mono- or polysubstituted aromatic hydrocarbons, for example toluene, o-, m-, p-xylene, ethylbenzene, chlorobenzene, or o-, m-, p-dichlorobenzene. Among these, very particular preference is given to o-, m- or p-xylene, chlorobenzene, o-, m-, p-dichlorobenzene or mixtures thereof.

When inert solvents are used in the reaction, their amount is generally up to 2000% by weight and preferably up to 1 to 1000% by weight, based on the total amount of the phthalic anhydride present and the phthaloyl chloride formed.

In order to obtain a maximum space-time yield, preference is given in the process according to the invention to using a minimum amount of or no solvent. Thus, preference is given to carrying out the reaction with a mixture which consists during the entire performance of the reaction to an extent of ≧80% by weight, preferably ≧90% by weight and more preferably to an extent of ≧99% by weight, of phthalic anhydride, chlorinating agent (I), catalyst (II) and intermediates, by-products and end products formed from these substances. Very particular preference is given to carrying out the reaction in the absence of an additional solvent.

“End products” refer to the phthaloyl chloride and sulfur dioxide or carbon dioxide products formed according to the main reaction equations

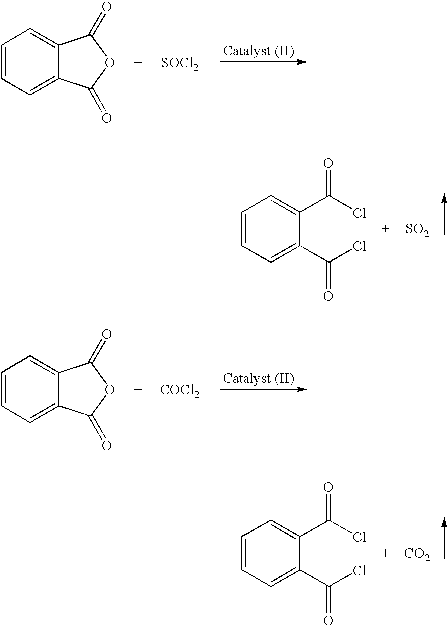

It should be emphasized at this point that the main amounts of sulfur dioxide or carbon dioxide formed escape in gaseous form from the liquid reaction mixture actually during the performance of the reaction.

“By-products” refer to the products which are formed by side reactions. An example thereof is 4-chlorophthaloyl chloride. In addition, by-products are also regarded as being the reaction products of the catalyst (II) used when they are present in the reaction mixture at the end of the inventive reaction, for example compounds which are formed by the reaction of triphenylphosphine or triphenylphosphine oxide with thionyl chloride or phosgene.

“Intermediates” refer to the products formed as intermediates during the reaction.

The process according to the invention is carried out at a pressure of from 0.01 to 10 MPa abs, preferably of from 0.05 to 5 MPa abs, more preferably of from 0.09 to 0.5 MPa abs and most preferably of from 0.09 to 0.2 MPa abs.

In addition, the process according to the invention is carried out at a temperature of from 80 to 200° C., preferably of from 100 to 180° C., more preferably of from 120 to 160° C. and most preferably of from 130 to 160° C.

After the desired amount of chlorinating agent has been added, the resulting reaction solution is generally left to continue to react under the reaction conditions for a certain time, generally from 30 minutes to 6 hours. In order to remove or deplete excess thionyl chloride or phosgene and their sulfur dioxide or carbon dioxide reaction products from the reaction solution, inert gas is subsequently generally passed through with intensive mixing (“stripping”).

The reaction effluent is generally worked up by the known methods. Preference is given to isolating the desired phthaloyl chloride by fractional distillation. The triphenylphosphine or triphenylphosphine oxide used as a catalyst (II) may, if required, be recovered by distillation and reused.

To achieve particularly high product purities, it is also possible to precipitate out any phthalic anhydride dissolved in the distilled phthaloyl chloride by adding a nonpolar solvent, for example cyclohexane, petroleum ether or toluene, and to remove it as a solid. The added solvent may then subsequently be distilled off again from the phthaloyl chloride.

In a preferred embodiment for the semicontinuous preparation of phthaloyl chloride using thionyl chloride, the desired amount of phthalic anhydride is introduced into a stirred tank with reflux condenser and heated to from about 130 to 160° C., so that a phthalic anhydride melt is present. About half of the desired catalyst (II) is added thereto with stirring. Alternatively, however, it is also possible to initially charge solid phthalic anhydride and about half of the desired catalyst (II) and melt them together. Subsequently, the addition of a liquid mixture of the thionyl chloride and the remaining catalyst (II) is commenced, and the addition rate is generally adjusted in such a way that the unconverted thionyl chloride boils gently under reflux. On completion of the addition of the thionyl chloride/catalyst (II) mixture, the reaction mixture is left to continue to react with further stirring for from about 0.5 to 6 hours and remaining sulfur dioxide is subsequently stripped out with nitrogen. Finally, the reaction mixture is fed to a distillation column in which first the excess thionyl chloride and then, preferably under reduced pressure, the phthaloyl chloride are distilled off.

In another preferred embodiment for the semicontinuous preparation of phthaloyl chloride using thionyl chloride or phosgene, the desired amount of phthalic anhydride is introduced into a stirred tank with reflux condenser and heated to from about 130 to 160° C., so that a phthalic anhydride melt is present. The desired catalyst (II) is added thereto with stirring. Alternatively, however, it is also possible to initially charge solid phthalic anhydride and the desired catalyst (II) and melt them together. Subsequently, the addition of thionyl chloride or phosgene is commenced, and the addition rate is generally adjusted in such a way that the unconverted thionyl chloride or phosgene boils gently under reflux. On completion of the addition of the chlorinating agent, the reaction mixture is left to continue to react with further stirring for from about 0.5 to 6 hours, and remaining sulfur dioxide or carbon dioxide is subsequently stripped out with nitrogen. Finally, the reaction mixture is fed to a distillation column in which first the excess chlorinating agent and then, preferably under reduced pressure, the phthaloyl chloride are distilled off.

The process according to the invention enables the preparation of phthaloyl chloride of high purity, in which only readily obtainable and industrially widely available phthalic anhydride, thionyl chloride or phosgene as chlorinating agents, and triphenylphosphine or triphenylphosphine oxide as catalysts have to be used and no coproducts which remain predominantly in the reaction mixture are formed. In addition, the process found leads even under mild temperatures and pressures to a high conversion of phthalic anhydride, a high selectivity for and a high yield of phthaloyl chloride, and especially to a high space-time yield. For instance space-time yields of distinctly above 60 g of phthaloyl chloride per liter of reaction volume and hour are achieved in the process according to the invention. In addition, the phthaloyl chloride product of value can be removed readily from the reaction mixture, and there is no risk of solid precipitation from the reaction mixture. The catalyst (II) which remains in the bottoms in the subsequent product distillation may be recovered therefrom if required or be disposed of or utilized thermally together with the remaining bottom product.

Definitions

Estimation of the Space-Time Yield

Since no reactor sizes are specified in the description of the experiments in the nearest prior art (in particular L. P. Kyrides, J. Am. Chem. Soc. 59, 1937, page 206 to 208 and DE-A 102 37 579), but the space-time yield is nevertheless a central quantity in the context of the present objective, the space-time yields were estimated. In order to enable comparability within the present patent application, both all space-time yields from the prior art and those from all inventive examples and comparative examples were estimated by the same rules. The estimation was as outlined hereinbelow:

The estimation is based on the formula

$$STY = \frac{{m}_{phthaloyl chloride}}{{V}_{reactor} \cdot t} .$$

m_{phthaloyl chloride} is the mass of phthaloyl chloride formed in g, V_{reactor} the estimated volume of a reactor which is technically viable for the specific reaction batch and t is the reaction time in hours including continued stirring time. To estimate the size of the technically viable reactor, it was assumed that it attains a maximum fill level of 60% by volume in the course of the reaction. Since the chlorinating agent was added continuously in all experiments in the nearest prior art and in the present patent application and had thus already been converted substantially during the addition, it was assumed that the maximum volume of the reaction mixture was present in each case at the end of the experiment. This volume was estimated from the volumes of the phthaloyl chloride formed, of the remaining residue between the phthalic anhydride used and the phthaloyl chloride formed, the catalyst used, the solvent used and the unconverted chlorinating agent remaining in the solution. In the latter case, it was assumed as an approximation that all of the excess of chlorinating agent remained in the solution and only the sulfur dioxide or carbon dioxide reaction products formed escaped.

### EXAMPLES

### Example 1

### Inventive

A solution of 6.5 g (0.025 mol) of triphenylphosphine in 150 g (1.26 mol) of thionyl chloride was added at 140° C. to a mixture of 74 g (0.5 mol) of phthalic anhydride and 6.5 g (0.025 mol) of triphenylphosphine within 3.5 hours. After 65 ml of this solution had been added, the internal temperature decreased to approx. 125° C. After the entire amount of the solution had been added, the mixture was stirred at 115° C. for a further 2 hours. Subsequently, remaining dissolved sulfur dioxide was stripped out with nitrogen. 162 g of homogeneous reaction effluent were obtained which, after vacuum distillation, gave 96 g of product. This contained 94.6 GC area % of phthaloyl chloride (0.447 mol, corresponding to 89% yield). The estimated space-time yield was about 72 g/l·h.

### Example 2

### Inventive

122 g (1.03 mol) of thionyl chloride were added at 140° C. to a mixture of 74 g (0.5 mol) of phthalic anhydride and 13 g (0.047 mol) of triphenylphosphine oxide within 4 hours. After 35 ml of this solution had been added, the internal temperature decreased to approx. 132° C. After the entire amount of the solution had been added, the mixture was stirred at 122° C. for a further 2 hours. Subsequently, remaining dissolved sulfur dioxide was stripped out with nitrogen. 159 g of homogeneous reaction effluent were obtained which, after vacuum distillation, gave 97 g of product. This contained 94.2 GC area % of phthaloyl chloride (0.45 mol, corresponding to 90% yield). The estimated space-time yield was about 75 g/l·h.

### Example 3

### Inventive

A solution of 6.5 g (0.023 mol) of triphenylphosphine oxide in 122 g (1.03 mol) of thionyl chloride was added at 140° C. to a mixture of 74 g (0.5 mol) of phthalic anhydride and 6.5 g (0.023 mol) of triphenylphosphine oxide within 4 hours. After 35 ml of this solution had been added, the internal temperature decreased to approx. 132° C. After the entire amount of the solution had been added, the mixture was stirred at 122° C. for a further 2 hours. Subsequently, remaining dissolved sulfur dioxide was stripped out with nitrogen. 161 g of homogeneous reaction effluent were obtained which, after vacuum distillation, gave 98 g of product. This contained 94.4 GC area % of phthaloyl chloride (0.456 mol, corresponding to 91% yield). The estimated space-time yield was about 76 g/l·h.

### Example 4

### Inventive

A solution of 4.5 g (0.016 mol) of triphenylphosphine oxide in 122 g (1.03 mol) of thionyl chloride was added at 142° C. to a mixture of 74 g (0.5 mol) of phthalic anhydride and 2.0 g (0.007 mol) of triphenylphosphine oxide within 5 hours. After 40 ml of this solution had been added, the internal temperature decreased to approx. 127° C. After the entire amount of the solution had been added, the mixture was stirred at 112° C. for a further 2 hours. Subsequently, remaining dissolved sulfur dioxide was stripped out with nitrogen. The resulting homogeneous reaction effluent (162 g) was initially freed of excess thionyl chloride at 200 hPa abs (200 mbar abs) and 35° C., and subsequently distilled at 9 hPa abs (9 mbar abs) and 137° C. The distillate (97.7 g), from which a few crystals of phthalic anhydride precipitated out in the course of standing, was admixed with 50 ml of cyclohexane. 5.3 g of precipitated phthalic anhydride were filtered with suction from the mixture and the cyclohexane was subsequently distilled off 92.0 g of product remained which contained 96.9 GC area % of phthaloyl chloride (0.44 mol, corresponding to 88% yield). The estimated space-time yield was about 66 g/1-h.

### Example 5

### Inventive

A solution of 30 g (0.11 mol) of triphenylphosphine oxide in 720 g (6.05 mol) of thionyl chloride was added at 140° C. to a mixture of 650 g (4.4 mol) of phthalic anhydride and 30 g (0.11 mol) of triphenylphosphine oxide within 8 hours. After 120 ml of this solution had been added, the internal temperature decreased to approx. 132° C. After the entire amount of the solution had been added, the mixture was stirred at 122° C. for a further 2 hours. Subsequently, remaining dissolved sulfur dioxide was stripped out with nitrogen. 1249 g of homogeneous reaction effluent were obtained which, after vacuum distillation, gave 826 g of product. This contained 94.2 GC area % of phthaloyl chloride (3.8 mol, corresponding to 87% yield). The estimated space-time yield was about 58 g/l·h.

Examples 1 to 5 show that, in the case of the inventive use of thionyl chloride as the chlorinating agent and of triphenylphosphine or triphenylphosphine oxide as the catalyst, space-time yields in the range from 58 to 76 g/1-h can be achieved.

### Example 6

### Inventive

181 g (1.82 mol) of phosgene were added at 135° C. to a mixture of 224 g (1.5 mol) of phthalic anhydride and 5.3 g (0.019 mol) of triphenylphosphine oxide within 5 hours. On completion of addition, the mixture was stirred at 113° C. for a further 0.5 hour. Subsequently, phosgene and dissolved carbon dioxide were stripped out with nitrogen at 60° C. 308 g of homogeneous reaction effluent were obtained and gave 300 g of product after vacuum distillation (1 hPa abs (1 mbar abs)). This contained 98.5 GC area % of phthaloyl chloride (1.48 mol, corresponding to 98.7% yield). The estimated space-time yield was about 131 g/l·h.

Example 6 shows that, in the case of the inventive use of phosgene as the chlorinating agent and of triphenylphosphine oxide as the catalyst, space-time yields of distinctly above 100 g/l·h can be achieved. It should be emphasized once again at this point that the space-time yields according to the prior art (U.S. Pat. No. 3,810,940 and DE-A 102 37 579) in the phosgenation of phthalic anhydride in the presence of N,N-dimethylformamide as a catalyst at from 31 to 52 g/l·h are only a fraction of the space-time yields of the process according to the invention.

### Example 7

### Inventive

164 g (1.66 mol) of phosgene were added at 126° C. to a mixture of 224 g (1.5 mol) of phthalic anhydride and 5.3 g (0.019 mol) of triphenylphosphine oxide in 306 g of chlorobenzene (275 ml) within 4 hours. On completion of addition, the mixture was stirred at 120° C. for a further 0.5 hour. Subsequently, phosgene and dissolved carbon dioxide were stripped out with nitrogen at 60° C. 609 g of homogeneous reaction effluent were obtained and were analyzed by gas chromatography. After arithmetic subtraction of the chlorobenzene solvent and of the catalyst used, the reaction effluent contained 97.9 GC area % of phthaloyl chloride in addition to 2.1 GC area % of unconverted phthalic anhydride, which corresponds to a yield of 97.9%. The estimated space-time yield was about 79 g/l·h.

### Example 8

### Comparative Example

72 g (0.73 mol) of phosgene were introduced at 70° C. into a mixture of 224 g (1.5 mol) of phthalic anhydride and 5.3 g (0.019 mol) of triphenylphosphine oxide in 306 g of chlorobenzene (275 ml). Vigorous reflux was observed, which pointed to the inadequate or only very insufficient conversion of the phosgene. The internal temperature rose to about 50° C. The experiment was terminated and the reaction mixture analyzed by gas chromatography. No formation of significant amounts of phthaloyl chloride could be detected, which corresponds to a space-time yield of 0 g/l·h.

Example 8 shows that a reaction temperature of 70° C. is insufficient for the reaction. By contrast, it was possible in Example 7 to achieve a very high conversion at a reaction temperature of 126° C. even in the presence of a solvent.

### Example 9

### Comparative Example

159 g (1.61 mol) of phosgene were added at 70° C., as per Example IV of U.S. Pat. No. 3,810,940, to a mixture of 224 g (1.5 mol) of phthalic anhydride and 1.4 g (0.019 mol) of N,N-dimethylformamide in 306 g of chlorobenzene (275 ml) within 5 hours. On completion of addition, the mixture was stirred at 70° C. for a further 1 hour. Subsequently, phosgene and dissolved carbon dioxide were stripped out with nitrogen at 60° C. 560 g of partly crystalline reaction effluent were obtained and were analyzed by gas chromatography. After arithmetic subtraction of the chlorobenzene solvent and the catalyst used, the reaction effluent contained 62.2 GC area % of phthaloyl chloride in addition to 37.6 GC area % of unconverted phthalic anhydride, which corresponds to a yield of only 62.2%. The estimated space-time yield was only about 41 g/l·h.

Example 9 shows clearly that the process conditions taught in U.S. Pat. No. 3,810,940 including the catalyst are absolutely unsuitable for achieving a high yield and a high space-time yield.

### Example 10

### Comparative Example

A solution of 26 g (0.075 mol) of Cyanex® 923 (mixture of various tri-C₆- to C₈-alkylphosphine oxides from Cytec Industries) in 603 g (5.1 mol) of thionyl chloride was added at 140° C. to a mixture of 450 g (3.0 mol) of phthalic anhydride and 26 g (0.075 mol) of Cyanex® 923 within 7 hours. On completion of addition, the mixture was stirred at 140° C. for a further 2 hours. Subsequently, dissolved sulfur dioxide was stripped out with nitrogen at 100° C. The reaction effluent which was partly crystalline was analyzed by gas chromatography. After arithmetic subtraction of the catalyst used, the reaction effluent contained only 19 GC area % of phthaloyl chloride in addition to 81 GC area % of unconverted phthalic anhydride, which corresponds to a yield of only 19%. The estimated space-time yield was merely about 16 g/l·h.

Comparative example 10 shows that there is a significant dependence in the process according to the invention on the type of catalyst used and, for example, a tri-C₆- to —C₈-alkylphosphine oxide leads to dramatically poorer results compared to triphenylphosphine oxide.

1-9. (canceled)

10. A process for preparing phthaloyl chloride which comprises reacting phthalic anhydride with a chlorinating agent (I) wherein said agent is thionyl chloride or phosgene in the presence of a catalyst (II) at a temperature of from 80 to 200° C. and a pressure of from 0.01 to 10 MPa abs, wherein said catalyst (II) is triphenylphosphine, triphenylphosphine oxide or a mixture thereof.

11. The process according to claim 10, wherein from 0.1 to 20 mol % of catalyst (II) based on the phthalic anhydride used are used.

12. Process according to claim 10, wherein the chlorinating agent (I) is used in a molar ratio of from 0.95 to 10 based on the molar amount of phthalic anhydride used.

13. Process according to claim 11, wherein the chlorinating agent (I) is used in a molar ratio of from 0.95 to 10 based on the molar amount of phthalic anhydride used.

14. The process according to claim 10, wherein the chlorinating agent (I) used is thionyl chloride.

15. The process according to claim 13, wherein the chlorinating agent (I) used is thionyl chloride.

16. The process according to claim 10, wherein the reaction is carried out with a mixture which consists, during the entire performance of the reaction, to an extent of ≧80% by weight of phthalic anhydride, chlorinating agent (I), catalyst (II) and intermediates, by-products and end products formed from these substances.

17. The process according to claim 15, wherein the reaction is carried out with a mixture which consists essentially of, during the entire performance of the reaction, to an extent of ≧80% by weight of phthalic anhydride, chlorinating agent (I), catalyst (II) and intermediates, by-products and end products formed from these substances.

18. The process according to claim 10, wherein the reaction is carried out with a mixture which consists essentially of, during the entire performance of the reaction, to an extent of ≧99% by weight of phthalic anhydride, chlorinating agent (I), catalyst (II) and intermediates, by-products and end products formed from these substances.

19. The process according to claim 17, wherein the reaction is carried out with a mixture which consists essentially of, during the entire performance of the reaction, to an extent of ≧99% by weight of phthalic anhydride, chlorinating agent (I), catalyst (II) and intermediates, by-products and end products formed from these substances.

20. The process according to claim 10, wherein the reaction is carried out in the absence of an additional solvent.

21. The process according to claim 15, wherein the reaction is carried out in the absence of an additional solvent.

22. The process according to claim 10, wherein the reaction is carried out at a pressure of from 0.05 to 5 MPa abs.

23. The process according to claim 21, wherein the reaction is carried out at a pressure of from 0.05 to 5 MPa abs.

24. The process according to claim 10, wherein the reaction is carried out at a temperature of from 120 to 160° C.

25. The process according to claim 23, wherein the reaction is carried out at a temperature of from 120 to 160° C.
